(19) United States
(12) Patent Application Publication
(10) Pub. No.: US 2026/0165665 A1
(43) Pub. Date: Jun. 18, 2026

(54) SYSTEMS AND METHODS FOR X-RAY DEVICE MONITORING USING AIR CALIBRATION

(71) Applicant: KONINKLIJKE PHILIPS N.V., EINDHOVEN (NL)

(72) Inventors: Carolina Maria RIBBING, AACHEN (DE); Heiner DAERR, HAMBURG (DE); Lester Donald MILLER, HUDSON, OH (US)

(21) Appl. No.: 19/129,345

(22) PCT Filed: Nov. 22, 2023
(86) PCT No.: PCT/EP2023/082627

§ 371 Date: May 13, 2025

Related U.S. Application Data
(60) Provisional application No. 63/428,751, filed on Nov. 30, 2022.

(51) Int. Cl. A61B 6/46; A61B 6/58

(52) U.S. Cl. CPC A61B 6/469; A61B 6/583; A61B 6/585

(57) ABSTRACT

A non-transitory computer readable medium (26) stores instructions executable by at least one electronic processor (20) to perform a method (100) of monitoring an X-ray imaging device (1). The method includes retrieving air calibration data (32) generated by at least one air calibration performed for the X-ray imaging device; deriving a diagnostic metric indicative of a problem with or status of the X-ray imaging device from the retrieved air calibration data; and outputting an alert or status indicator (30) indicative of the problem with or status of the X-ray imaging device based on the derived diagnostic metric.

### FIELD

The following relates generally to the medical imaging arts, X-ray imaging arts, X-ray calibration arts, X-ray maintenance arts, and related arts.

### BACKGROUND

For predictive maintenance of medical imaging systems, there is a need for sensitive component monitoring to make a timely replacement possible without unplanned downtime. To this end, sensor-based monitoring can be implemented in order to provide early detection of X-ray tube failure. Often, a tube add-on equipped with sensors for monitoring is utilized.

Current systems can use an auxiliary unit coupled to the tube housing. The auxiliary unit carries tube data, e.g., from the tube control unit and additional sensors. Estimation of the remaining tube lifetime is achieved by a model in a remote computer, operating on the data transmitted by the auxiliary unit (the prediction can also possibly be done online in the auxiliary unit. For tube anode bearing failure prediction, current systems can also include a diagnostic circuit as sensor for increased anode motor load. An accelerometer is used to measure the acceleration of the gantry and the anode rotation frequency and acoustic, voltage and current sensors are used to monitor the anode drive motor.

Furthermore, for prediction of tube bearing failures, accelerometers, microphones and vibration sensors have been suggested, such as a circuit board device to detect vibration in the tube housing during an anode rotor coast down to zero. Also, incorporation of sensors to detect acoustic noise and vibration for tube bearing failures can be used. Other systems can use sensors for position, vibration, angle, and temperature measurements for an X-ray tube predictive fault indicator. Tube vibration data is transformed, and a notification is sent whenever a certain number of diagnostic frequencies are detected.

Other components besides the X-ray tube can similarly benefit from early detection of indications of component failure. The X-ray detector array is typically constructed as a two-dimensional (2D) array of individual X-ray detectors. Early detection of degradation of performance of an X-ray detector can enable timely replacement without unplanned downtime. In the case of computed tomography (CT) imaging scanner, a rotating gantry carries the X-ray tube and X-ray detector array. The gantry is a complex mechanical system that can experience degradation such as bearing wear or the like that can introduce wobble or other rotational imperfections that can adversely impact image quality and gantry safety. Again, early detection of gantry problems can be beneficial. However, adding sensors to monitor these additional components introduces further complexity to the X-ray imaging device, increases the costs and also additional components prone to fail.

The following discloses certain improvements to overcome these problems and others.

### SUMMARY

In some embodiments disclosed herein, a non-transitory computer readable medium stores instructions executable by at least one electronic processor to perform a method of monitoring an X-ray imaging device. The method includes retrieving air calibration data generated by at least one air calibration performed for the X-ray imaging device; deriving a diagnostic metric indicative of a problem with or status of the X-ray imaging device from the retrieved air calibration data; and outputting an alert or status indicator indicative of the problem with or status of the X-ray imaging device based on the derived diagnostic metric.

In some embodiments disclosed herein, an X-ray imaging system includes an X-ray imaging device and a controller configured to control the X-ray imaging device to acquire an air calibration; determine gain normalization factors for X-ray detectors of the X-ray imaging device using the air calibration; control the X-ray imaging device to acquire an image of an associated subject using the determined gain normalization factors; and store air calibration data generated by at least one air calibration. An electronic processor is programmed to retrieve the stored air calibration data; derive a diagnostic metric indicative of a problem with or status of the X-ray imaging device from the retrieved air calibration data; and output an alert indicative of the problem with or status of the X-ray imaging device based on the derived diagnostic metric.

In some embodiments disclosed herein, a non-transitory computer readable medium stores instructions executable by at least one electronic processor to perform a method of monitoring an X-ray device. The method includes retrieving air calibration data generated by at least one air calibration performed for the X-ray device; deriving a diagnostic metric indicative of a problem with or status of the X-ray device from the retrieved air calibration data; and outputting an alert indicative of the problem with or status of the X-ray device based on the derived diagnostic metric.

One advantage resides in monitoring an X-ray device using air calibration data instead of additional sensor on the X-ray device.

Another advantage resides in monitoring failure of an X-ray tube, detector, high-voltage generator, high-voltage wiring, bowtie filter, collimator, and/or gantry using calibration data.

Another advantage resides in predicting a failure time of an X-ray device to determine an optimal servicing time for an X-ray device, thereby reducing a down time of the X-ray device.

Another advantage resides in reducing delayed or cancelled X-ray examinations.

Another advantage resides in decreased financial losses for a medical institution based on accurately determining a predicted failure date of an X-ray device.

Another advantage resides in recommending a date for a next air calibration process, reducing time spent with unnecessary air calibrations as well as ensuring that they are made often enough to guarantee proper calibration of each imaging scan.

Another advantage resides in reduction of elimination of cancellation of patient scans because of unplanned downtime

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an X-ray imaging device in accordance with the present disclosure.

FIG. 2 diagrammatically illustrates an X-ray device monitoring method using the X-ray imaging device of FIG. 1.

### DETAILED DESCRIPTION

In the normal course of operation of an X-ray imaging system, an air calibration scan is done, for example on a weekly basis for CT scanners. The air calibration scan is done with nothing loaded in the gantry (hence, an “air” scan) but with the system otherwise set up for an imaging session. For example, any X-ray filters that would be used in imaging a patient are in place (e.g., dose filter, wedge filter, bow-tie filter, et cetera). The air calibration scan is used to generate normalization factors for normalizing measured intensities during patient imaging.

The following discloses leveraging this existing air calibration data to monitor imaging system health. Advantageously, this does not involve adding any new sensors to the system, does not include collecting any new (type of) data, and does not entail any additional work on the part of imaging system personnel. Rather, the air calibration is compared with one or more previous air calibrations to detect changes that may be indicative of a developing problem with the imaging system. As such air calibration is already performed, the data collection framework is already present and in regular use. Furthermore, as no patient is loaded in the gantry, but rather only air is scanned, the air scans do not contain any patient information, which would otherwise constitute a privacy issue for use of the data for system monitoring. As one example of leveraging the air calibration scan for device monitoring, if the detector signals for all the X-ray detectors of the detector array decrease over time, this can indicate a problem with the X-ray tube. On the other hand, if the detector signals from a subset of the detectors corresponding to a single detector module decrease over time this likely indicates that detector module is failing. As yet another example, in the case of a CT scanner the air calibration data are acquired for each angle position because the detected intensity depends on the gantry angle. Heavy components on the gantry deform the gantry and by this the distance of tube and detector change and thus the detected detector signal intensities, If the variation in detector signal as a function of gantry angle increases over time, this could indicate a gantry problem such as bearing fatigue and/or imbalance in the gantry.

In some embodiments, the X-ray imaging system health analyses can be performed each time an air calibration scan is done. The analyses could be done locally, and the results presented on the display of the imaging device controller along with other air calibration scan results. In another embodiment, the air calibration data could be uploaded to the vendor server along with other machine log data and the analysis performed at the remote server or in a cloud server, with any problems being reported to a Remote Service Engineer (RSE) or the like. In some embodiments, if a local analysis of the air calibration data detects an urgent problem this could be output as an urgent alert.

In some embodiments, early detection of a component problem may be based on comparison of a current air scan with a previous air scan to detect a change or may be based on comparison of two or more successive air scans to detect a trend in the data. This approach presupposes that the previous air scan data are stored. However, an air calibration scan generates a large amount of data. For example, in one nonlimiting illustrative example of a commercial CT scanner, each detector takes measurements over a large number of gantry positions (or “views,” e.g., 2320 views in some commercial CT scanners), and there are many thousands of detectors in the X-ray detector array. Consequently, in the normal course of operation the air calibration scan data are typically overwritten regularly since they are only used once for generating the normalization factors. To implement the disclosed X-ray imaging system health monitoring, all or some subsets of the air calibration data are stored longer term, either locally or remotely. In one approach, every Nth calibration is stored. Additionally or alternatively, the raw air calibration data can be processed to extract a smaller set of features that are used in the system health monitoring, which can reduce the amount of data stored over the longer term. For example, only a subset of the 2320 views of the illustrative example may be stored, e.g., one out of every ten views going around the 360° rotation can be stored thereby reducing the size of the stored dataset to one-tenth of the acquired raw data.

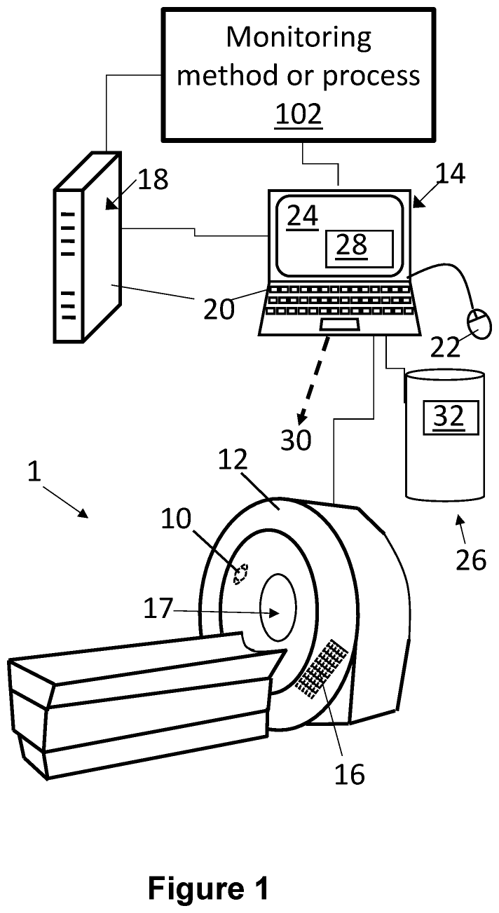

With reference to FIG. 1, an illustrative medical system including a medical device 1 is shown. The medical device 1 can be an X-ray imaging device such as, for example, a computed tomography (CT) imaging device (as shown), or a C-arm imaging device such as are sometimes used for cardiac imaging, or an image guided therapy (IGT) system employing X-ray imaging, a fluoroscopic imaging device, a digital radiography (DR) imaging device, or other X-ray imaging device that utilizes X-ray imaging (hereinafter referred to as an “X-ray imaging device” or variants thereof). More generally, the medical device 1 can be any medical device having a component for which a calibration can be performed to monitor the component, such as a linear accelerator (LINAC). As shown in FIG. 1, the medical X-ray device 1 can include a component, such as an X-ray tube 10 shown by partial removal of a gantry 12 of the CT scanner 1 in FIG. 1. The diagrammatically shown X-ray tube 10 is a simplified representation modern commercial X-ray tubes used in X-ray imaging devices often include additional components such as a grid whose geometry and electrical bias can be used to control the shape, focus, intensity, or other characteristics of the X-ray beam, and such components may introduce additional X-ray tube performance variables such as the grid voltage. The X-ray device 1 also includes or is in operative communication with a device controller 14 configured to control operation of the X-ray imaging device 1.

The X-ray imaging device 1 further includes an X-ray detector 16 that is configured to detect the X-ray radiation emitted by the X-ray tube 10 after the X-rays pass through an examination region 17. During operation to acquire imaging data of a patient or other imaging subject (not shown), that imaging subject is disposed in the examination region 17. On the other hand, during an air calibration scan the examination region 17 is unloaded, so that the examination region 17 contains only air. As shown in FIG. 1, the detector 16 typically comprises a detector array, and the X-ray tube 10 emits a cone beam of X-rays that pass through the examination region 17 and thence impinge on the detector array 16. The detector 16 is also in electronic communication with the device controller 14, such as a workstation computer, or more generally a computer. Images produced by the X-ray device 1 via the X-ray radiation generated by the X-ray tube 10 are processed by the device controller 14 and/or another electronic processing device 18, which may for example be implemented as a server computer 18 as shown. The illustrative CT scanner 1 employs tomographic imaging in which the X-ray tube 10 and detector 16 rotate together around an imaging subject to acquire a three-dimensional (3D) image of the subject. In other types of X-ray imaging devices these components may be fixed in position rather than revolving around the imaging subject, and hence provide a two-dimensional (2D) image. In a C-arm configuration such as is sometimes used in X-ray imaging systems for cardiac imaging, image-guided therapy (IGT), or other clinical applications, the X-ray tube 10 and the detector 16 can be moved on robotic arms or the like to different vantage points (called “views”) around the patient to (for example) provide clinically significant views of the heart, or in an IGT to provide a chosen view of an interventional procedure.

The electronic processing device 18 is an optional component that may include a workstation, a server computer (as shown) or a plurality of server computers, e.g., interconnected to form a server cluster, cloud computing resource, various combinations thereof, or so forth, to perform more complex computational tasks. For example, in a common configuration the device controller 14 is provided for controlling the imaging device 1 to perform image acquisition, and also to record machine log data (e.g., operating parameters of the X-ray imaging device 1, alerts, errors, or the like generated by the X-ray imaging device 1, and so forth; in some embodiments disclosed herein the log data may also include the air calibration data or a subset thereof or features derived therefrom); while the server 18 is connected via a hospital network and/or the Internet to occasionally receive updates of the machine log data (which in some embodiments disclosed herein may include the air calibration data or subset thereof or features derived therefrom). The device controller 14 and/or server 18 includes typical components, such as an electronic processor 20 (e.g., a microprocessor), at least one user input device (e.g., a mouse, a keyboard, a trackball, and/or the like) 22, and a display device 24 (e.g., an LCD display, plasma display, cathode ray tube display, and/or so forth).

The electronic processor 20 is operatively connected with one or more non-transitory storage media 26. The non-transitory storage media 26 may, by way of nonlimiting illustrative example, include one or more of a magnetic disk, RAID, or other magnetic storage medium; a solid-state drive, flash drive, electronically erasable read-only memory (EEROM) or other electronic memory; an optical disk or other optical storage; various combinations thereof, or so forth; and may be for example a network storage, an internal hard drive of the workstation 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a visualization of a graphical user interface (GUI) 28 for display on the display device 24.

The disclosed imaging system is further configured as described above to perform a method or process 100 of monitoring a medical device. Although described herein as the medical device being the X-ray device 1 and a component being one of the X-ray tube 10, the gantry 12, and/or the detector 16, the method 100 can apply for any suitable component of any suitable medical device for which a deviation from a performance of the component compared to an expected performance of the component (e.g., calibration data) occurs. The non-transitory storage medium 26 stores instructions which are readable and executable by the at least one electronic processor 20 to perform disclosed operations including performing the monitoring method or process 100. Depending on the processing capability of the device controller 14 and the availability of a server or other additional computing resources 18, the monitoring 100 of the health of the X-ray imaging device 1 can be performed using the imaging device controller 14, the server 18, or both. In one suitable example, the device controller 14 controls the X-ray imaging device 1 to acquire an air calibration and processes the air calibration data to generate normalization factors for normalizing measured intensities during patient imaging; while the X-ray device monitoring method 100 may be performed by the imaging device controller 14 or the server 18. In some examples, the method 100 may be performed at least in part by cloud processing.

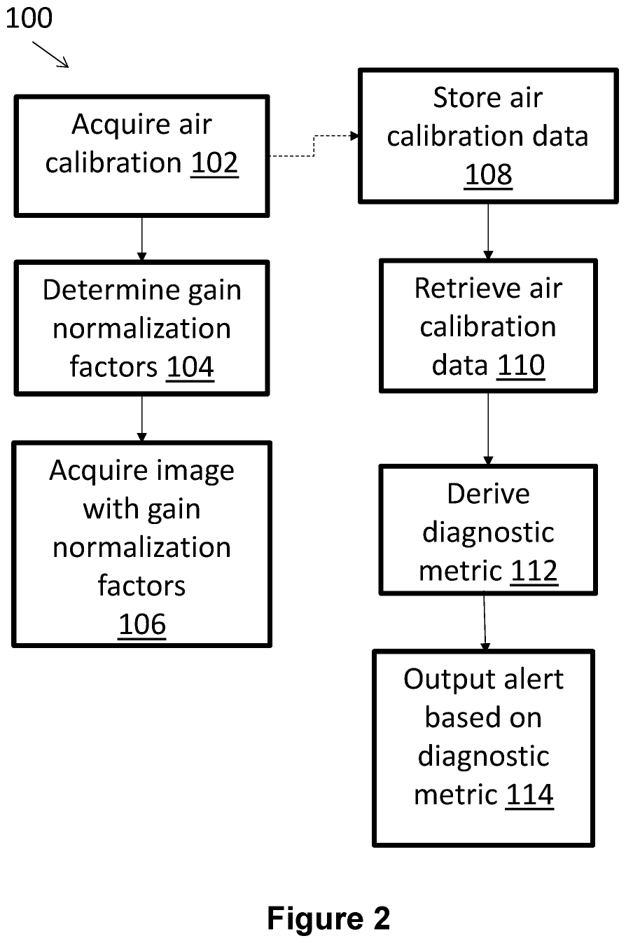

With reference to FIG. 2, an illustrative embodiment of an instance of the monitoring method 100 is diagrammatically shown as a flowchart. To begin the method 100, a calibration process is performed. At an operation 102, the controller 14 is configured to control the X-ray imaging device 1 to acquire an air calibration. In the air calibration, the examination region 17 of the X-ray imaging device 1 is not loaded, so that the X-rays emitted by the X-ray tube 10 pass through air in the examination region 17 and thence are detected by the X-ray detector array 16. Thus, the air calibration data do not contain any patient information. While the examination region 17 is unloaded, depending on the detailed air calibration being performed one or more X-ray beam shaping components may be disposed in the path of the X-rays, such as a bow-tie filter (also referred to as a wedge filter), collimator, and/or so forth. For example, if performing an air calibration to calibrate for a patient imaging sequence that normally uses a bow-tie filter, then that bow-tie filter is suitably installed during the collection of the air calibration data. A set of scan parameters (e.g., kV, mA, slice thickness, rotation time, focal spot, wedge/filter/collimator settings, and so forth) needs a corresponding air calibration performed with the same parameter settings. Since no patient is loaded, the intensities measured by the detector of the detector array 16 ideally should be the same across the detector array 16, and in the illustrative case of the CT scanner 1 should also be the same for each of the (e.g., 2320) views that are acquired as the gantry 12 rotates the X-ray tube 10 and detector array 16 together. In practice however, nonuniformities may be present due to variations in detector sensitivity across the detector array 16, spatial variation in the X-ray beam intensity, imperfections or displacements in the bow-tie filter, wedge filter, or other X-ray beam shaping component(s), and/or so forth.

At an operation 104, the controller 14 is configured to determine gain normalization factors for the X-ray detectors 16 using the acquired air calibration. The gain normalization factors compensate for the nonuniformities such as variations in detector sensitivity across the detector array 16, spatial variation in the X-ray beam intensity, imperfections in the bow-tie filter, wedge filter, or other X-ray beam shaping component(s), and/or so forth. The gain normalization factors for the X-ray detectors are suitably stored in the non-transitory storage medium 26 for use in patient imaging. The air calibration 102, 104 may be performed on a weekly basis or other designated calibration interval to periodically update the gain normalization factors.

At an operation 106, the controller 14 is configured to control the X-ray imaging device 1 to acquire an image of a subject using the gain normalization factors determined in the latest occurrence of the air calibration 102, 104. In this way, the various nonuniformities such as variations in detector sensitivity across the detector array 16, spatial variation in the X-ray beam intensity, imperfections in the bow-tie filter, wedge filter, or other X-ray beam shaping component(s), and/or so forth, are compensated by the latest gain normalization factors determined in the latest occurrence of the operation 104. While FIG. 2 illustrates a single instance of an image acquisition 106, it will be appreciated that many such image examinations may be performed in the week (or other time interval) between successive repetitions of the air calibration 102, 104.

While the air calibration 102, 104 is principally performed to update the gain normalization factors, as recognized herein the air calibration data acquired in each occurrence of the operation 102 (that is, in each air calibration) also provides data from which information about the health of the X-ray imaging device 1 can be extracted. In particular, significant changes in corresponding measured values of the air calibration between different repetitions of the air calibration 102, 104 can be analyzed to provide an early warning of component failure. In some examples, differences in air calibrations from the same date can be analyzed and used as early warning (such as for comparing calibrations for different sets of settings or on comparing signals over the detector 16 or over the various views). For example, roughening of the focal track of the anode will result in spectrum hardening which will manifest itself as enhanced intensity variation over the various detector slices (due to the variation in the amount of anode material transversed by the radiation on leaving the anode). Such roughening can be followed by comparing air calibrations taken for different slice thickness and over time. Comparison of air calibration data for different parameters (e.g., kV, mA, field of view, and focal spot settings) can provide information on focal spot changes, e.g., spot position. Comparison of data from air calibrations at different gantry speeds and gantry tilt angles can provide information on gantry bearing and potential gantry wobble, gantry imbalance, gantry components coming loose, and anode bearing status. Potential scout scan (surview) air calibrations in AP (anterior-posterior) and LR (left-right) direction can further be used for anode bearing status monitoring.

If two, three, or more repetitions of the air calibration 102, 104 are analyzed, trend lines can be extracted, from which for example a remaining life of a component may be estimated. Hence, at an operation 108, the controller 14 is configured to store air calibration data 32 generated by at least one air calibration in the non-transitory storage media 26 of the electronic processing device 18. In some examples, the controller 14 is configured to store air calibration data generated by at least two air calibrations performed at different times. In some examples, the controller 14 is configured to store air calibration data generated by at least three air calibrations performed at different times so as to provide more detailed trend information. In some embodiments, the operation 108 stores the entire raw air calibration data generated by the operation 102. In other embodiments, the operation 108 performs feature extraction, data selection, data compression, or other processing of the raw air calibration data to reduce the otherwise large size of the raw calibration dataset to reduce storage requirements. In another example, the air calibration data 32 (or some subset thereof or features derived therefrom) is stored as additional log data in log files in the non-transitory storage media 26 of the electronic processing device 18. For example, the air calibration data 32 may be stored in log files that store other data commonly automatically logged during operation of the imaging device (e.g., kV, mA, slice thickness, rotation time, focal spot, wedge/filter/collimator settings, and so forth).

To implement the imaging device health monitoring, the method 100 includes an operation 110 in which the stored air calibration data 32 is retrieved from the non-transitory storage media 26. As previously mentioned, the detector signal data stored at the operation 108 may have been processed to generate values for a set of features from the air calibration data and with the values stored for the set of features—in this case, the stored values for the set of features are suitably retrieved in the operation 110.

At an operation 112, in addition to performing the calibration (i.e., the gain normalization in operation 104), a diagnostic metric indicative of a problem or a status (e.g., predicted time to failure for a component) with the X-ray imaging device 1 is derived from the retrieved air calibration data 32. The operation 112 leverages the air calibration data 32 to, in addition to actively calibrating the detector gains, also passively detect a problem or status of the imaging device 1. As used herein, the term “diagnostic metric” (and variants thereof) refers to a measurement which can be related to a component or function of the X-ray imaging device 1 or a binary “yes/no” item related to a component of the X-ray imaging device 1. In some embodiments, air calibration data 32 for two or more air calibrations performed for the X-ray imaging device 1 at different times can be retrieved, and the diagnostic metric is derived based on a difference or trend over time in the air calibration data 32 being indicative of the problem with the X-ray imaging device 1. In a similar manner, differences and trends can be derived based on air calibration data from the same point in time but from different configurations of the X-ray imaging device 1 (i.e., different calibration settings or scanner settings, e.g., kV, mA, slice thickness, gantry speed, tilt, C-arm direction, C-arm trajectory, axial scan direction, filter, collimator and wedge settings, etc.). In some embodiments, the controller 14 is configured to store air calibration data 32 generated by at least two air calibrations performed with different configurations of the X-ray imaging device 1, and the electronic processor 18 is programmed to derive the metric indicative of the problem with the X-ray imaging device 1 based on a difference between the air calibrations or values derived therefrom.

In some embodiments, the diagnostic metric is indicative of a problem with the X-ray tube 10. For example, the diagnostic metric comprises an estimated time remaining until end-of-life (EOL) of the X-ray tube 10 being less than a threshold time. In another example, the diagnostic metric comprises a change in intensity, spectrum, X-ray intensity, or focal spot position of the X-ray tube 10. In some embodiments, the diagnostic metric is indicative of a problem with the X-ray detector 16. For example, the diagnostic metric comprises an estimated time remaining until end-of-life (EOL) of the X-ray detector 16 being less than a threshold time. The diagnostic metric could also comprise a difference between detector pixels or modules, indicating that certain modules need to be inter-exchanged or replaced, or indicating that certain parts of the detector 16 are bleached or contaminated with, for example, spilled contrast.

In some embodiments, the diagnostic metric is indicative of a problem with the gantry 12. For example, the diagnostic metric comprises a change in a linearity, a stability, an angle, or a position of the gantry 12. Various approaches can be used to determine what type of component issue is indicated by a difference or trend in the air calibration data (or features derived therefrom). For example, if all detector intensities are decreasing over time at a similar rate (and similarly for all views or positions of the X-ray detector in the case of a CT or C-arm where the detector moves), this may be inferred to indicate an issue with the X-ray tube 10 since the single X-ray tube 10 illuminates all the detectors 16. On the other hand, if only some X-ray detectors of the array 16 exhibit a decrease in intensities, then this may be inferred to indicate an issue with those X-ray detectors, or with positioning or with spectral hardening of the X-ray tube 10. If all the detectors exhibiting the decrease in intensities belong to a single X-ray detector module or group of modules, then this more particularly may enable inference that the problem is with that module or group of modules. As yet another example, there can be a cyclical variation in measured intensities coinciding with the 360° rotation of the gantry 12. If this variation is observed to increase over time, then this may be inferred to indicate a problem with the stiffness, stability or positioning of the rotating gantry 12 or a displacement of a gantry component. The forces on gantry components vary with gantry speed, tilt, and position, which can provide additional information on gantry component and anode bearing status. Other problems which may be detected are e.g., misalignment of patient couch, misalignment of the anti-scatter grid on the detector, misalignment of collimator or collimator parts, cracks in the bow-tie filter, and so forth. Advantageously, such inferences are not mutually exclusive, e.g. an observed overall decrease in intensities coupled with a larger decrease for a certain detector subset of the detector array 16 could enable inference of both a problem with the X-ray tube 10 and a problem with the detectors subset; and the cyclical variation with rotation of the gantry 12 can also be observed and used for additional component and function characterization. These are merely examples and should not be construed as limiting.

At an operation 114, an alert or status indicator 30 indicative of the problem or status with the X-ray imaging device 1 determined from the air calibration data 32 in the step 112 is output based on the derived diagnostic metric. In some examples, the alert 30 includes a recommendation to replace a component (i.e., the X-ray tube 10, the detector 16, and so forth) of the medical device 1. In some examples, certain combinations of settings (e.g., high-power tube stings) can be avoided, such as exchanging two detector modules, updating a system software, performing additional calibration, or conditioning. In other examples, the alert 30 is output on the display device 24 of the electronic processing device 18, and/or on a display device of a remote monitoring workstation. To do so, the remote monitoring workstation receives alerts from a medical device fleet that includes the medical device 1, and a representation of the alerts from the medical device fleet is output on the GUI 28. In another example, the log files and/or the defined subset data or features derived therefrom for the different scan parameters of the air calibration data 32 can be transferred to a remote service center for analysis of the alert or status indicator 30 indicative of the problem or status with the X-ray imaging device 1. In some embodiments, the log data transmitted to the remote service center is stored at the remote service center that provides servicing of the X-ray imaging device 1, and the operations 110, 112, and 114 are performed at the remote service center. In this approach, the air calibration data 32 (or the subset thereof or the features derived therefrom) is retrieved from the transmitted log data that is stored at the remote service center, and the deriving of the diagnostic metric and the outputting of the alert or status indicator 30 are performed at the remote service center.

In some embodiments, in a context in which a device malfunction could be attributable to different root causes, the operation 112 may additionally derive information about the root cause of a detected problem, to provide differential diagnosis indicating which component is failing to produce a detected problem. For example, a reduction in X-ray beam intensity over time could be due to a failing X-ray tube or could be due to a failing high voltage (HV) generator powering the X-ray tube 10. These differential failure modes may be detectable in the trend of air calibration data (and/or detectable in air calibrations performed with different configurations of the X-ray imaging device 1), for example using a machine learning (ML) component trained on labeled historical examples of air calibration data over time for X-ray systems with failing X-ray tube and with failing HV generator, so that the trained ML component can differentiate between these two component failure cases.

### EXAMPLE

CT scanners create three-dimensional (3D) images by reconstruction of X-ray attenuation data acquired over multiple view angles. Various corrections are applied to the raw attenuation data to obtain artifact-free images accurately representing the scanned patient. For example, proper calibration of each scan is required to correct for variations in scanner settings, X-ray tube output, and attenuation characteristics. To this end, regular (i.e., weekly) “air calibrations” are made with an empty bore to get the initial intensity per detector pixel (including e.g., bow-tie filter). Each set of scan parameters (kV, mA, slice thickness, gantry rotation time, focal spot, wedge/filter/collimator settings) will need a corresponding air calibration at the very same parameter settings. The resulting air calibration vectors indicate the relative efficiency and gain of the detector pixels and variation in the X-ray beam intensity across the radiation field. They are used to normalize the attenuation data acquired during patient scans. After such normalization, the detector signal is uniform over all detector pixels. This procedure is similar in all X-ray imaging systems where imaging is used for quantification to some extent.

The disclosed X-ray system uses any image correction changes over time in order to monitor system and X-ray tube status, wear and deterioration. The image correction contained in the air calibration vectors will change with time and gantry position and the changes will contain indications of imperfections like wear, leakage and imbalance. The available information content is rather large due to the presence of air vectors for various sets of scan parameters. Both air vector data from direct measurements and from delta manipulation can be used.

Normally, the air vector readings are logged and normalized with the reference detector reading, and thereafter compressed to save memory on the host computer. A common compression method is to use the mean values for a certain number 10<N<20 of sectors of the gantry, e.g., to obtain N=16 air vectors per gantry rotation from the original one per view acquired.

The logged raw, the normalized data, or the normalized compressed air vectors can be used by the disclosed system for monitoring. Measures like distance to previous/first/earlier air vector(s), vector size, norm, SNR, and corresponding measures for the frequency transformed vectors can be used. Also, the bare reference detector signal or its inverse can be used for monitoring (difference to previous signals, size, norm, SNR).

Typical air calibration data can include a voltage of the X-ray tube 10, a current of the X-ray tube 10, a collimation coverage, a focal spot size, a resolution, a wedge dose filter, a gantry rotation time, an extra filter, and so forth.

The disclosed system uses non-static image correction data to monitor X-ray device 1 imperfections, perturbations, or changes in the form of material fatigue, component wear, and leakage, causing e.g., vibrations, wobble, noise, and changes in e.g., image resolution, image quantification, X-ray intensity, focal spot characteristic such as position, uniformity, and homogeneity.

The acquired air calibration data 32 is detailed low-level detector data for analysis through multiple scales, from detailed to macro, including for example identifying bad detectors modules/pixels for direct service notification (e.g., based on deviations, rate of change of values, and noise); monitoring various types of global detector changes (e.g. temperatures in the gantry 12 or the detector 16); monitoring variations as function of gantry angle (e.g. for detecting balance changes and loosening of parts, other variations outside the norm could indicate other types of failures); monitoring the status of a plastic bowtie filter (e.g. detecting (developing) cracks); monitoring beam hardening and intensity changes as the X-ray tube 10 ages, monitoring the detectors 16 (i.e., identifying shadowing and misalignment); recommendation of a date for the next air calibration (e.g. if detector gains are changing); and so forth.

The information contained in the air calibration vectors can be used for system status monitoring in several ways, such as using the distance per detector pixel (e.g. Euclidean, Hamming, Minkowski, Jaccard, Dice, and so forth) to the last corresponding air vector as measure of system change/wear/deterioration since last the air calibration; using the distance per pixel to a previous air vector as measure of system wear/deterioration since last air calibration; using air vector range, standard deviation, mean, median, etc.; using air vector size, or (absolute) norm; using air vector noise, e.g. the signal-to-noise ratio (SNR) or the integrated noise spectrum; using the Hounsfield or CT number of air as computed from the air vector values; using reference-detector normalized or non-normalized air vectors, or only the reference detector signal; using a subset of air vector elements or all of them; using the 10-20 compressed air vectors or the original number of vectors per gantry rotation (non-compressed); using the fast Fourier transform (FFT) of the air calibration signal in combination with any method listed above to follow the development of frequency peaks over time and rotation (this only allows frequency response of e.g. 10 Hz at 0.5 s gantry rotation time); using spatial variation over the patient detector, e.g. comparing the values at the left, the right and at the center; and so forth. The beginning of the sampling can be critically examined to characterize e.g., overshoot and settling of the kV and mA, which can give information on high-voltage generator status. These can be quantified, characterized, and followed in ways similar as described above for the air vectors. The two DFS focal spots can be compared. The difference per pixel between the two focal spots can be calculated and compared to previous tables to detect changes in the focal spot deflection unit. It could even be that air vector measures (e.g., an absolute Euclidean norm) can be used to detect a problem without referencing back to previous or original installation air calibrations. This could be the case e.g., in examining differences between DFS settings, focal spot size, different kV settings, etc.

Any cross comparisons can be used for monitoring and early problem detection. The calibrations can be made at various scan conditions with combinations of focal spot size, gantry speed, kV and filtration. Any conceivable differences/distances/ratios can be computed and used for monitoring of the tube and system status. For instance, the Euclidean distance between the air vectors taken at high and low gantry speed can be used as indication of imbalance or anomaly. In the same way, the air vectors from large vs small focal spot can be used to detect focal spot issues, etc.

In imaging systems employing a rotating gantry for 3D imaging, gantry rotation produces a sinusoidal variation of the detector signal from each pixel of the patient detector and the reference detector. A sine signal from the reference detector and from pixels at various locations on the patient detector can be compared, especially their amplitudes. When differences develop, this indicates a gantry or tube problem. Any other corrections that vary over the lifetime of the X-ray tube, e.g., off-focal radiation correction, can also be employed for monitoring.

To save storage on a CT scanner host computer, the compressed air vectors are continuously overwritten with the most recent vectors. To still make comparisons to previous vectors possible, several ways are conceivable. Previous air vectors may be saved on the host or remotely in a cloud/computer/server. Alternatively, only certain features computed from previous air vectors, or only the difference from the corresponding previous vector, may be saved locally or remotely.

For use of Fast Fourier Transform (FFT) of detector signals, the development of peaks (i.e., appearance, disappearance, frequency, amplitude or AUC over time) could be used to monitor the X-ray tube 10 and other system components (gantry 12, generator, detector 16, fans, cooling, pump, heat exchanger, cables, and so forth). For the X-ray tube 10, anode rotation and wear (FFT peak height or area), focal spot degradation (e.g., position), bearing rotation and wear, various drive frequencies like mains and supply voltages with ripples (gantry drive, power blocks, grid, filament, tube, anode drive, detector), sampling rates, slips and duty cycles can be monitored. Peaks corresponding to mechanical vibration of the tube emitter (coil or flat emitter) can change over time due to material evaporation i.e., filament wear. On the anode side, peaks emanating from anode rotation will increase in amplitude with e.g., anode imbalance and wobble. For ball as well as spiral groove bearings, bearing status and wear can also be reflected in detector signal FFT spectra, e.g., as frequency shift, higher harmonics of the rotational frequency, due to e.g., lubricant loss, track wear, and anode motor drive slip. For slotted anodes, the slots may give rise to additional diagnostic possibilities. The positions of anode and filament peaks can shift slightly due to temperature changes, e.g., reflecting the momentary tube use. However, since air calibration is most often done directly after warm-up, temperature changes should have limited impact.

Detected deviations from previous air calibrations can also indicate a degradation of the overall image quality. In this case, a suitable maintenance action could consist of scheduling and conducting a full system calibration by a service engineer at a point in time to have minimal impact on normal workflow.

The disclosed methods can be understood as hand-crafted features. These features can be used to train a neural network or other (machine learning) algorithm to detect failures. The corresponding training data would consist of extracted potential features and actual failures and/or system issues. Instead of using analytic pre-defined detection methods or algorithms (hand-crafted features), all data can be fed into a (deep) learning algorithm (e. g. a convolutional neural network) together with the failure/system issues as labels (outcomes).

The disclosed systems and methods are applicable for CT, cone beam CT/C-arm systems characterized by e.g., X-ray tubes with ball bearings that are under too high cost pressure for additional sensors. Use in other imaging systems like any cardiovascular system, PET-CT, SPECT-CT, MR-CT, flat X-ray, mammography, and linearly scanning systems for e.g., luggage scanning is also contemplated. As soon as regular air calibration of an X-ray imaging system is employed, the calibration data can be leveraged for system monitoring.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing instructions executable by at least one electronic processor to perform a method of monitoring an X-ray imaging device the method comprising:
retrieving air calibration data generated by at least one air calibration performed for the X-ray imaging device;
deriving a diagnostic metric indicative of a problem with or status of the X-ray imaging device from the retrieved air calibration data; and
outputting an alert or status indicator indicative of the problem with or status of the X-ray imaging device based on the derived diagnostic metric.

2. The non-transitory computer readable medium of claim 1, wherein the diagnostic metric is indicative of a problem with or status of an X-ray tube of the X-ray imaging device.

3. The non-transitory computer readable medium of claim 1, wherein the diagnostic metric is indicative of a problem with or status of an X-ray detector of the X-ray imaging device.

4. The non-transitory computer readable medium of claim 1, wherein the method further comprises:
determining X-ray detector gain normalization factors using the air calibration.

5. The non-transitory computer readable medium of claim 1, wherein the diagnostic metric is indicative of a problem with or status of a gantry of the X-ray imaging device.

6. The non-transitory computer readable medium of claim 1, wherein:
the diagnostic metric comprises an estimated time remaining until end-of-life (EOL) of a component of the X-ray imaging device being less than a threshold time.

7. The non-transitory computer readable medium of claim 1, wherein:
the retrieving includes retrieving air calibration data for two or more air calibrations performed for the X-ray imaging device at different times; and
the diagnostic metric is derived based on a trend over time in the air calibration data being indicative of the problem with the X-ray imaging device.

8. The non-transitory computer readable medium of claim 1, wherein:
the retrieving includes retrieving air calibration data for two or more air calibrations performed for the X-ray imaging device with different configurations of the X-ray imaging device; and
the diagnostic metric is derived based on a difference between the two or more air calibrations or values derived therefrom being indicative of the problem with the X-ray imaging device.

9. The non-transitory computer readable medium of claim 1, wherein the method further comprises:
processing detector signal data acquired during the at least one air calibration to generate values for a set of features from the air calibration data and storing the values for the set of features;
wherein the retrieving of the air calibration data generated by the at least one air calibration comprises retrieving the stored values for the set of features.

10. The non-transitory computer readable medium of claim 1, wherein the method further comprises:
controlling the X-ray imaging device to acquire the air calibration data generated by the at least one air calibration; and
storing the air calibration data obtained by the controlling.

11. The non-transitory computer readable medium of claim 1, further comprising:
storing log data generated by the X-ray imaging device including operating parameters of the X-ray imaging device and further including the air calibration data or a subset thereof or features derived therefrom; and
transmitting the stored log data to a remote service center;
wherein the retrieving of the air calibration data comprises retrieving the air calibration data or the subset thereof or the features derived therefrom from the stored log data.

12. The non-transitory computer readable medium of claim 1, further comprising:
storing log data generated by the X-ray imaging device including operating parameters of the X-ray imaging device and further including the air calibration data or a subset thereof or features derived therefrom; and
transmitting the stored log data to a remote service center and storing the transmitted log data at the remote service center;
wherein the retrieving of the air calibration data comprises retrieving the air calibration data or the subset thereof or the features derived therefrom from the transmitted log data that is stored at the remote service center, the deriving and outputting being performed at the remote service center.

13. The non-transitory computer readable medium of claim 1, wherein outputting the alert includes:
outputting the alert as a recommendation to replace a component of the medical device.

14. The non-transitory computer readable medium of claim 13, wherein the remote monitoring workstation receives alerts from a medical device fleet that includes the medical device and the method further comprises:
presenting a representation of the alerts from the medical device fleet on a graphical user interface (GUI) provided on a display device.

15. An X-ray imaging system, comprising:
an X-ray imaging device
a controller configured to:
control the X-ray imaging device to acquire an air calibration;
determine gain normalization factors for X-ray detectors of the X-ray imaging device using the air calibration;
control the X-ray imaging device to acquire an image of an associated subject using the determined gain normalization factors; and
store air calibration data generated by at least one air calibration; and
an electronic processor programmed to:
retrieve the stored air calibration data;
derive a diagnostic metric indicative of a problem with or status of the X-ray imaging device from the retrieved air calibration data; and
output an alert indicative of the problem with or status of the X-ray imaging device based on the derived diagnostic metric.

16. The X-ray imaging system of claim 15, wherein the controller is configured to store the air calibration data as features extracted therefrom.

17. The X-ray imaging system of claim 15, wherein the controller is configured to store air calibration data generated by at least two air calibrations performed at different times, and the electronic processor is programmed to derive the metric indicative of the problem with or status of the X-ray imaging device based on a trend over time in the air calibration data.

18. The X-ray imaging system of claim 15, wherein the controller is configured to store air calibration data generated by at least two air calibrations performed with different configurations of the X-ray imaging device and the electronic processor is programmed to derive the metric indicative of the problem with the X-ray imaging device based on a difference between the air calibrations or values derived therefrom.

19. A non-transitory computer readable medium storing instructions executable by at least one electronic processor to perform a method of monitoring an X-ray device the method comprising:
retrieving air calibration data generated by at least one air calibration performed for the X-ray device;
deriving a diagnostic metric indicative of a problem with or status of the X-ray device from the retrieved air calibration data; and
outputting an alert indicative of the problem with or status of the X-ray device based on the derived diagnostic metric.

20. The non-transitory computer readable medium of claim 19, wherein:
the retrieved air calibration data includes X-ray imaging device calibration data generated by calibration of the X-ray imaging device and
the diagnostic metric is derived from the X-ray imaging device calibration data.
